(19) United States
(12) Patent Application Publication
(10) Pub. No.: US 2003/0082618 A1
(43) Pub. Date: May 1, 2003

(54) Methods for detecting genetic aberrations

(76) Inventors: Guangshan Li, Corning, NY (US); Uwe R. Muller, Waukegan, IL (US); Yijia Bao, DesPlaines, IL (US); Xinying Xie, Painted Post, NY (US); Nalini Raghavachari, Painted Post, NY (US)

Correspondence Address: CORNING INCORPORATED, SP-TI-3-1, CORNING, NY, 14831

(21) Appl. No.: 10/271,193

(22) Filed: Oct. 15, 2002

Related U.S. Application Data
(60) Provisional application No. 60/329,590, filed on Oct. 15, 2001.

(51) Int. Cl. C12Q 1/68; C12P 19/34; B05D 3/00

(52) U.S. Cl. 435/6; 435/91.2; 427/2.11

(57) ABSTRACT

Methods and DNA microarray devices for detecting or measuring genetic aberrations or changes in genomic DNA using comparative genomic hybridization (CGH) techniques and gene-expression assays are provided.

### CLAIM OF PRIORITY

The present Application claims benefit of priority from U.S. Provisional Application No. 60/329,590, filed on Oct. 15, 2001, the entire content of which is incorporated herein.

### FIELD OF INVENTION

The present invention relates to DNA microarray technology. More particularly, the invention concerns methods and DNA microarray devices for detecting or measuring genetic aberrations or changes in genomic DNA using comparative genomic hybridization (CGH) techniques, and on the RNA level, using expression assays in a high throughput format.

### BACKGROUND

Genetic variations occur in many heritable and non-heritable genetic diseases. For example, alterations in the expression of tumor-suppressor genes and oncogenes have been linked to gene deletions and amplifications, respectively. Moreover, many well-known fetal syndromes are characterized by the gain or loss of gene functions caused by the addition or deletion of parts of the genome, leading to aneuploidies. Thus, detection and mapping of gene copy number changes provides an approach for associating aberrations with disease phenotype and for localizing critical genes, both of which are important for the fundamental understanding of genetic diseases and their diagnosis. Typically, two genomes are compared by labeling total genomic DNA with different fluorescent dyes and then co-hybridizing these complex probes to metaphase spreads. This enables mapping as well as semi-quantitative measurement of copy number abnormalities (CNAs).

Fluorescence in situ hybridization (FISH) is a well-established and precise technique to determine gene sequence copy numbers in individual cells with a resolution better than 30 Kb, but requires a priori knowledge of the target sequence to be analyzed. This can be an inconvenience, if not a limit, to the ability to study certain genes. Comparative genomic hybridization (CGH), on the other hand, permits a global analysis of the entire genome, which can detect chromosomal regions that have CNAs. Comparative genomic hybridization in metaphase spreads have rather low resolutions, on the order of 10-15 Mb. This problem has led researchers to turn to DNA chip technology (so-called genosensor-based (gCGH) or matrix-based CGH), which combines the high resolution of FISH with the inherent multiplex capability of CGH.

Use of CGH technique in DNA microarrays, especially in genosensor-based CGH (gCGH) applications, requires targets derived from BAC, PAC, or P1 clones, which contain inserts of genomic DNA fragments, human or other, on the order of less than one hundred to several hundred kilobases. It was the size of these inserts, typically ranging from 80 Kb to 150 Kb, and their spacing along the human genome, that determines the resolution and sensitivity. Large target sequences of greater than about 100 Kb contributes to a significant gain in resolution. The use of large cDNA index arrays, it is believed, would have the potential for high-resolution genome-wide analysis of gene copy number and high-throughput screening for diagnosis of genetic diseases. Several complications, however, exist with such an approach. It is believed that increased complexity of the targets results in higher signal strength with a concomitant loss in resolution. At least theoretically, the genetic complexity of a target spot on the microarray is directly proportional to the number of probe nucleotides that it can bind, as long as the target DNA concentration is not rate limiting. Absolute signal strength alone, however, does not determine assay sensitivity. In practice, a host of other parameters, such as probe complexity and concentration, label type and density, hybridization time and temperature, and a variety of other factors related to detection mechanism and instrumentation are of equal importance.

The primary reason for using large genomic clones in the first development phases of CGH technique was the need for sensitivity and robustness, since the complexity of the genome is at least about a 100 fold larger than the complexity of the transcriptome. When compared to gene expression assays, the amount of probe available for each target in a gCGH assay is, on average, only about 1% of that in a gene expression assay. As a consequence, it is believed that successful application of cDNA arrays in gCGH is rare, and single copy gene changes can not be measured reliably with this technology. Expecting higher specificity and increased power to discriminate between very similar genes and splice variants, the trend is now shifting toward oligomer targets for expression applications.

When compared to cDNA arrays, oligonucleotide-based arrays have become a preferred device for gene expression profiling, since they are relatively easy to develop, less expensive, and provide better specificity. Use of oligonucleotide-based arrays to measure gene copy numbers, however, will require an increase in detection sensitivity of an order of magnitude, relative to cDNA microarrays. To date, it is believed that, since replacement of cDNA targets with oligonucleotide sequences would cause an even further decrease in the expected sensitivity, it would be difficult to perform gCGH with oligonucleotide-based arrays without reducing the genome complexity and without amplifying the target sequences by polymerase chain reaction (PCR) or other amplification methods beforehand. In the past, only a few researchers have tried to use oligonucleotide arrays to assess gene copy number after the probes were prepared by multiplex PCR amplification using 10-50 primer pairs. But, it is well known that during amplification of different genes PCR amplification of probes can generate biases. More importantly, multiplex PCR is possible only for a small number of genes with a set of specific primers.

Deviation from normal physiology is generally accompanied by a multitude of histological and biochemical changes. These changes in turn are predominantly dictated by gene expression changes in the cell. The regulation of gene activity in cells could either be a physio-pathology or cause for a disease. Until recently, fine changes in the expression of genes associated with certain diseases have been studied using lower throughput approaches such as differential display PCR, subtractive hybridizations, sequencing of EST libraries or SAGE elements. Although, these approaches have identified disease genes, in general, these techniques are believed to be laborious and often tend to be insensitive.

DNA-microarray technology is a powerful tool that allows the simultaneous analysis of a large number of nucleic acid hybridizations in a rapid and efficient manner (see e.g., Beisker, W., Dolberate, F. and Gray J. W., CYTOMETRY 8, pp. 235-9, 1987, An Improved Immunocytochemical Procedure for High Sensitivity Detection of Incorporated Bromodeoxyuridine; Bolzan, A. D., Paez, G. L., Bianchi, M. S., and Bianchi, N. O., CANCER GENETIC CYTOGENET. 15, pp. 166-170, 2000, Analysis of Telomeric Repeats and Telomerase Activity in Human Colon Carcinoma Cells with Gene Amplification; Brown P O, Botstein D., NAT. GENETICS 21 (1 Suppl), pp. 33-7, 1999, Exploring the New World of the Genome with DNA Microarrays). It appears now that it is almost impossible without the help of this technology to gain valuable insights in a high-throughput manner in the areas of target discovery, mechanism of drug action, genes and pathways involved in various cellular responses and tumor classification. A major obstacle of expression array hybridization, however, has been the inability to detect low abundance transcripts. Further, broader application of microarrays is limited by the amount of RNA required. This appears to be a major drawback for profiling gene expression on biopsy specimens or blood cells. Current widely adopted protocols typically allow detection of a single gene copy among 100,000 transcripts (Cai, S., Shen, D., and Wang, J., CHIN. MED. J. 112, 242-245, 1999, Gene Deletion and Carrier Detection in the Family of Becker Muscular Dystrophy by Short Tandem Repeat Sequence Polymorphism).

Interestingly, this sensitivity and specificity still lags behind that of a classical northern blot performed with 5-10 μg of T.RNA leading to missed rare gene induction or suppression effects in mammalian tissues. In addition, artifacts arising due to spurious deposits of contaminants on the surface and the fluorescent dye used in the preparation of probes produce artifactual gene induction/suppression results, which warrant a need to improve hybridization specificity.

Currently, there are several hybridization protocols all aimed to achieve a high signal to noise ratio. These methods basically differ in the assay parameters, such as either the composition of the hybridization buffer, reaction temperature, blocking agents, or the amount of RNA required to prepare labeled probes for hybridization. In general, formamide based hybridizations at ˜42° C. have been reported to work better than aqueous hybridization solutions at ˜65° C. as it favors high signal to noise ratio but the kinetics of hybridization are slower than in aqueous solution. Blocking agents that are commonly used to minimize the noise include Denhardt's reagent, SDS, Sheared Salmon sperm DNA, tRNA, Cot-1 DNA, and Poly-A RNA to bind to T-rich sequences.

A cDNA or oligonucleotide array normally needs 20-100 μg of total RNA for gene expression profiling. The FISH technique has a very low resolution of about 10-15 mb. The low resolution of large genomic clone-based CGH, such as of Pac or P1, still limits the use of this technology. Also, cDNA-based CGH have low sensitivity. Oligonucleotide-based CGH needs probe amplification. Therefore, given the foregoing, a need exists for a better and simpler protocol for performing gene expression assays and CGH techniques, which enables mapping and semi-quantitative measurement of copy number abnormalities (CNAs). The present invention satisfies this need for 1) a highly sensitive gene-expression-profiling array that can use only a few micrograms (˜2 μg) of total RNA, and 2) a high resolution, high sensitivity oligonucleotide-based CGH array, without probe amplification.

### SUMMARY OF THE INVENTION

The present invention details methods for determining relative gene copy numbers and profiling gene expression with high sensitivity, specificity, and reproducibility. One method is for performing RNA expression analysis. The method comprises several steps. First, reduce the autofluorescence of a substrate containing an array of biomolecules. The autofluorescence reducing step further comprises: providing a substrate having a first surface with a functional group for binding of an unmodified biomolecule, arraying a set of target biomolecules onto the first surface, and treating at least a portion of the first surface with a reducing agent. Then, apply an amount of either total RNA or mRNA, without amplification, ranging from 50-200 μg of total RNA and about 10 μg or less of mRNA, preferably about 2-5 μg. Label by fluorescent means, such as Cy3 or Cy5 dyes, a number of cDNA probes, which are generated by reverse transcription from either the total RNA or mRNA using either random primers, semi-random primers, anchored dT, or a combination thereof. Pretreat the first surface of the substrate with a blocking reagent to reduce non-specific binding of said cDNA probes to the target biomolecules. By means of a predetermined hybridization mixture, increase the local concentration of cDNA probes to promote hybridization efficiency and optimizing stringency to promote specificity. Hybridize a pool of complementary cDNA probes to the target biomolecules. Again, treat the first surface of the substrate with the blocking reagent during the hybridization. Image the array on the first surface with different wavelengths to determine the relative fluorescence signal intensity of hybridized cDNA probes on target biomolecules, followed by analysis of the fluorescence ratio. Preferably, the amount of either total RNA or mRNA applied, without amplification, is about 5 μg or less. The amount of mRNA applied, without amplification, can be about 0.5 μg or less. The CDNA probes have a length of between about 70 (100) bases to about 3 (7) kilobases, preferably, about 100 (400) bases to about 1 (5) kilobases.

A second method is for performing comparative genomic hybridization (CGH) on a high quality oligonucleotide-based DNA array. The method employs an extremely sensitive assay. This method permits the use of total genomic DNA without the need to perform sample amplification, such as by PCR. The method is anticipated to allow genome-wide analysis of gene copy numbers with a sufficient sensitivity and reliability, such as that required for the detection of genetic changes that are based on the loss or gain of a single copy sequence. The method comprises the following steps. First, reduce autofluorescence on a substrate containing an array of oligonucleotides. The autofluorescence reducing step further comprises: providing a substrate having a first surface with a functional group for binding unmodified oligonucleotides; arraying a set of target oligonucleotides onto the first surface; treating at least a portion of the first surface with a reducing agent. Then, label genomic DNA (gDNA) probes from a test sample and a reference sample with a first fluorescent dye and a second fluorescent dye, respectively. Apply an amount of gDNA, without amplifying said gDNA, of about 10 μg or less. Pretreat the first surface of the substrate with a blocking reagent to reduce non-specific binding of the gDNA probes to the target oligonucleotides. Using a predetermined hybridization mixture, increase local concentration of gDNA probes to promote hybridization efficiency and optimize stringency to promote specificity. Hybridize the gDNA probes to the target oligonucleotides. During hybridization, again treat the first surface of the substrate with the blocking reagent. Image the relative fluorescence intensity of the first and second fluorescent dyes to determine the relative ratio of the first and second fluorescent dyes to represent a copy number of a gene. Preferably, the amount of gDNA applied, without amplifying said gDNA, is about 1-10 μg. More preferably, the amount of gDNA applied, without amplifying said gDNA, is about 5 μg or less. The oligonucleotides have a length of about 15 bases to about 110 bases. Preferably, the oligonucleotides have a length between about 40 or 50 bases to about 100 bases.

Furthermore, the invention includes a cDNA- or oligonucleotide-based microarray for determining relative gene copy numbers. In particular, the oligonucleotide-based array can perform analysis of genetic abrerrations using total genomic DNA without need to amplify, according to the method for performing comparative genomic hybridization (CGH).

### BRIEF DESCRIPTION OF FIGURES

FIG. 1 presents dose responses of gene copy number changes as measured by means of CGH performed on an oligonucleotide array. A: Genomic DNAs from the test and the reference samples were labeled with Cy3- and Cy5-dCTP, respectively. Different amounts of Cy5-labeled MYC DNA were spiked into each assay mixture, which contained equal amounts of Cy3- and Cy5-labeled placenta DNA probes. This probe mixture was hybridized to the oligonucleotide array. The normalized ratio of Cy5 and Cy3 fluorescence intensities of a gene was used to represent the copy number of the gene. The solid line was drawn by regression analysis with a computer program. B: Equal amounts of Cy3- and Cy5-labeled placenta DNA probes were hybridized on the same kind of oligonucleotide array. The normalized ratio of Cy5 and Cy3 fluorescence intensities for each gene was plotted against Cy5 net relative fluorescence unit (RFU).

FIG. 2A is an image of an oligonucleotide microarray spotted with the genes MYC and PVT1.

FIG. 2B is a graph that represents gene copy numbers in colo320 using the microarray of FIG. 1A, with the location of each oligonucleotide of the gene given along the x-axis and the gene copy number along the y-axis

FIG. 3 is an image of an oligonucleotide-based CGH with four subgrids used to measure gene copy number.

FIG. 4A is an image of a microarray used according to a method of the present invention to perform gene expression profiling with total RNA without amplification.

FIG. 4B is a graph of the results from the microarray of FIG. 4A, wherein the location of each gene given along the x-axis and the expression ratio along the y-axis.

FIG. 5 is an image after SYBR green staining and shows an uniform distribution of array spots on glass slides produced by means of a high-throughput contact-printing technology developed at Coming Inc.

FIG. 6 shows array hybridization results with different primers on cancer arrays.

FIG. 7 demonstrates that labeling with different kind of primers can give very different but expected hybridization specificity.

FIG. 8 shows that dextran sulfate improves the assay sensitivity.

FIG. 9 shows that pre-selected assay condition according to the present invention can provide hybridization data with a high level of confidence, with a good correlation of Cy5- and Cy3-signal intensity and <9% of CV of ratios across the slide.

FIG. 10 demonstrates that the marker gene for vitamin D24 hydroxylase was detected with less than about 5 μg of total RNA input.

FIG. 11 shows that for a slide hybridized with about 0.5 μg of total MCF RNA, 65% of targets have a signal/background ratio greater than 1, and 43% of targets have a signal/background ratio greater than 2.

FIG. 12 confirms the microarray assay results performed with RT-PCR, which is the gold standard in the microarray field for evaluation of microarray data.

### DETAILED DESCRIPTION OF THE INVENTION

Considering all of the variables and parameters, mentioned above, that affect the measured gene expression levels in a typical microarray analysis, the present invention attempts to further improve microarray technology. We report a highly sensitive and specific array system for the differential gene expression analysis emphasizing on ways to eliminate technology artifacts from limited amounts of RNA.

The present invention entails methods that provide for improved sensitivity and allow for the use of probe concentrations of about 80 ng or less of sample DNA per μL of hybridization solution with either CDNA array or high quality oligonucleotide array platforms. The methods enable researchers to perform RNA expression analysis, using either an amount of about 25 μg to about 10 μg or less of total RNA or approximately 500 ng to 100 ng or less of MRNA. Alternatively workers may employ array-based CGH, using total genomic DNA, without the need to perform amplification. It is anticipated that the method can monitor gene copy numbers in a genome-wide analysis with sufficient sensitivity and reliability as that required for the detection of genetic changes that are based on the loss or gain of a single copy sequence.

As delineated above, the methods for determining relative gene copy numbers comprise a number of steps. Common to both methods, the autofluorescence of the substrate, containing biomolecules (e.g., cDNA, oligonucleotides, or other species), is reduced or set virtually to zero. Treating the substrate with a reducing agent, selected from the group consisting of hydrogen and hydrides, accomplishes this task. More particularly, the reducing agent includes a borohydride, preferably a sodium borohydride. Other potential reducing agents may include sodium cyanoborohydride and copper sulfate. Additionally, this kind of treatment can effectively eliminate autofluorescence of the unmodified oligonucleotides bound to the substrate surface. Autofluorescence reduction of substrates is described in greater detail in U.S. patent application Ser. No. 09/925,808, entitled “Treatment of Substrates for Immobilizing Biomolecules,” filed in the name of Y. Bao et al., on Aug. 9, 2001, the content of which is incorporated herein by reference in its entirety.

Because of the demand for higher sensitivity and accuracy in detection, researchers previously have needed to use PCR to amplify probe sequences. Multiplex PCR, however, is only possible for a small number of genes with specific primers of about 10-50 primer pairs, is rather cumbersome, and can easily introduce biases for the probe sequences. The invention, in contrast, uses less amounts of nucleic material, without the need for PCR or other amplification techniques, than previously possible. This feature contributes to savings in cost and time and the use of rare or scarce sample materials and reagents. An important consideration is the amount of sample material required for analysis. For example, in clinical applications, since tumor specimens are getting smaller as diagnoses are being made earlier, the inventive methods can work with samples obtained by biopsy. Until a robust and reliable total genome PCR amplification method that does not distort the sequence ratios becomes available, workers in the field will have to rely on non-amplified sample materials. To that end, assays conducted according to the present inventive methods have achieved satisfactory results with as little as about 3 μg of tumor DNA in hybridizations carried out overnight, without the aid of signal amplification or hybridization systems that are designed to overcome the barriers-of diffusion. Preliminary results in the laboratory suggest that significantly greater sensitivity is possible. The inventive methods may also be applied to other functions, such as analysis of individual clones in tumor tissue or the analysis of perhaps single fetal cells obtained from maternal blood.

Furthermore, the present invention allows the direct use of genomic DNA to measure gene copy numbers, and is a more reliable and simpler way for oligonucleotide array-based CGH. Significantly, the method expands the resolution of gCGH technology by a factor of greater than 10, and makes the detection of small sequence changes—perhaps even single base-pair changes—feasible on a genome-wide scale.

To promote better hybridization efficiency, according to the invention, local concentration of either cDNA or gDNA is increased by density or a volume exclusion technique. A macromolecule polymer, such as dextran sulfate or polyethylene glycol, is mixed with an amount of nucleic sample in an aqueous solution. The polymer absorbs the water thus increasing the concentration of biomolecules. According to the present invention, polymer concentration needs to be controlled, maintaining it at a level lower than about 10% of conventional usage with nitrocellulose membranes. The polymer concentration should be about 8.5% or less, preferably between about 6% and 4% or 3%. A good blocking reagent, such as bovine serum albumin (BSA) or fat-free dry milk, can reduce the likelihood of high nonspecific binding, which the addition of dextran sulfate tends to increase. Furthermore, to promote greater specificity, according to the invention, stringency is optimized by means of a predetermined hybridization solution mixture, with a diluted or reduced salt concentration, and different amounts of organic solvents, such as formamide, dimethyl sulfoxide (DMSO), or ethylene glycol. The salt concentration of hybridization solutions should be maintained at or less than conventional concentration levels of about 5×-4×SSC. Preferably, the salt concentration should be about 2×SSC or less.

Using CGH assay technique in DNA microarrays, especially in genosensor-based CGH (gCGH), total genomic DNA from a test sample and reference DNA (from normal tissues) is labeled with fluorescent dyes, like either Cy3 or Cy5 dye. The Cy3- and Cy5-labled probes were co-hybridized to the microarray. The ratio of Cy3 and Cy5 fluorescence intensities for each spotted clone sequence is approximately proportional to the ratio of the copy numbers of the corresponding sequences in the test and the reference genomes.

It is believed that the present invention can provide the requisite sensitivity and robustness to make possible efficient high resolution genome-wide scanning with CDNA or oligonucleotide-based arrays. It is envisioned that the present invention will afford rapid and cost efficient testing for gene copy number changes in pathological tissues. This advantageous feature is likely to result in a considerable increase in understanding of genetic disease. The ideal test would allow whole genome scanning at gene or low level resolution or below with a single chip and a minimum amount of sample material. The sensitivity of array based CGH is already sufficient for detection of low-level amplifications and aneuploidies if the tumor tissue is not too diluted with normal cells and if most of the cells carry the mutation. Even though in very homogeneous tissues, such as cell lines, certain leukemias, or tissues from patients with hereditary disease, arrays of large genomic clones have been feasible in detecting single copy deletions or additions, the source of CDNA clones for CDNA microarray is limited. Production of oligonucleotide arrays will not be limited by the CDNA clone resources. Other virtues and features of the invention are further expounded and clarified in the following examples.

### EXAMPLES

As used in this description of the invention, the term “target” refers to the DNA or biomolecules bound on the microarray substrate, so as to be consistent with the CGH and gCGH literature, but persons in the art will realize that an alternative nomenclature has emerged, in which the DNA on the microarray substrate is referred to as the “probe.”

I. According to the present invention, oligonucleotide arrays dramatically reduced the interference of repetitive sequences during hybridization. Results obtained according to the present method indicate that one can detect more accurately genetic aberrations than even comparable results obtained from large genomic clone arrays, even of those from CDNA arrays, which typically contain poly-A sequences. Moreover, oligonucleotide arrays dramatically expand the resolution power of CGH, potentially even as far down as to a few base pairs. This feature alone was extremely important since many genetic diseases are the result of relatively small sequence changes, which currently remain undetected in cytogenetic laboratories.

Oligonucleotides from disease genes and some normal genes were designed and synthesized. For example, the oligonucleotides were printed on a support surface such as gama-aminopropylsilane (GAPS) treated glass slides. Genomic DNAs from the test and the reference samples were labeled with fluorescent dyes, Cy3- and Cy5-dCTP, respectively. The Cy3- and Cy5-labeled probes were hybridized to the oligonucleotide array. The ratio of Cy3 and Cy5 fluorescence intensities of a gene was used to represent the copy number of the gene.

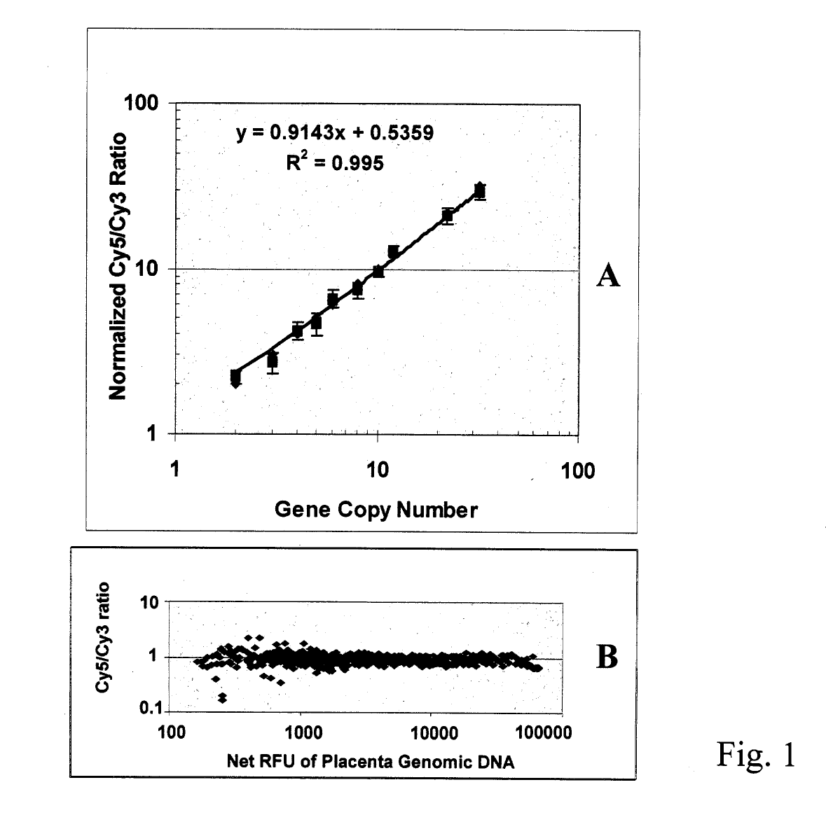

We measured the dose response of the present oligonucleotide array-CGH system by labeling placenta DNA separately with either Cy3-dCTP or Cy5-dCTP, and then mixing approximately equal amounts to simulate a test and reference sample, as is typical for CGH type experiments. Different amounts of Cy5-labeled MYC DNA were spiked into this probe mixture before hybridizing to the array. The ratio of net fluorescence intensities for each target spot was averaged between the repeat arrays and then normalized as described in the legend to FIG. 1. As that figure shows, in otherwise normal diploid DNA the addition of two or more copies of a given gene can be detected with a high degree of confidence. For a single copy addition (e.g. a trisomie) detection is also possible (FIG. 1A). The data from the hybridization with equal amounts of Cy3- and Cy5-labeled placenta DNA probes indicated that all of the oligonucleotides had a ratio close to 1 with a CV of 19% (FIG. 1B). Furthermore, approximately 95% of all target spots have a positive signal at the 99% confidence level, i.e. the net signal is larger than 3 standard deviations of the background. The key contributing factors for achieving this level of sensitivity with oligonucleotide arrays is 1) the intrinsic low autofluorecence of the slide substrate used, 2) high quality oligonucleotide microarrays, 3) the chemical reduction of autofluorescence of the slide coating and the target spots prior to hybridization, and 4) our optimized protocols for labeling, hybridization and post hybridization treatments

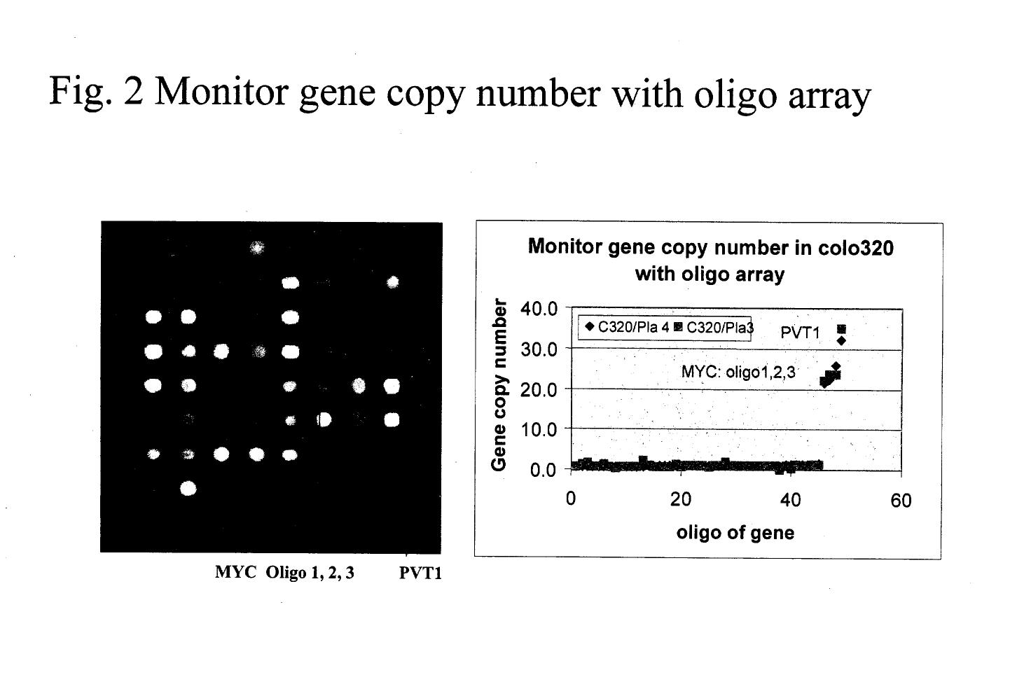

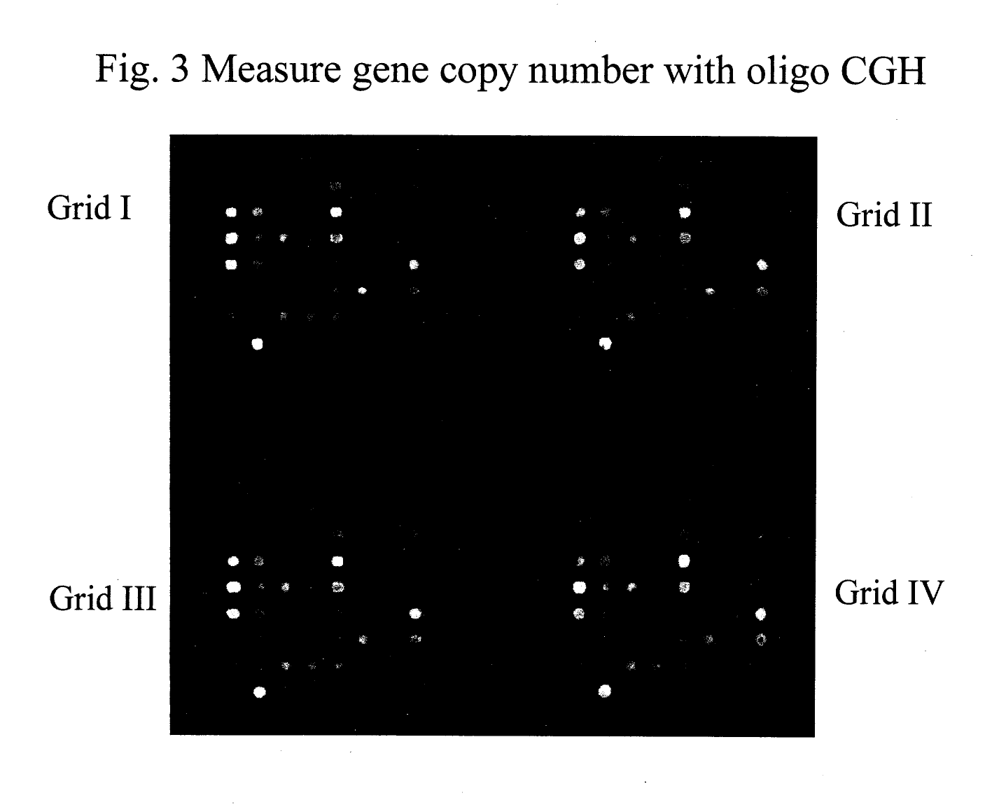

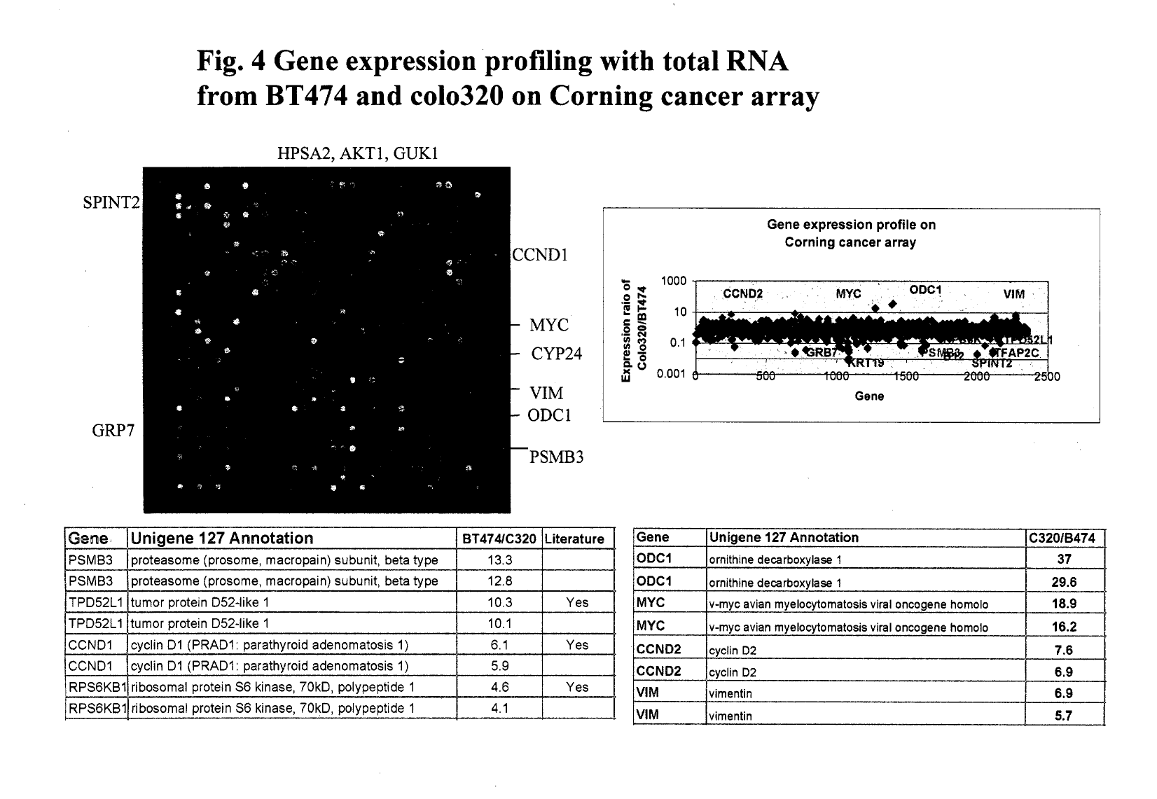

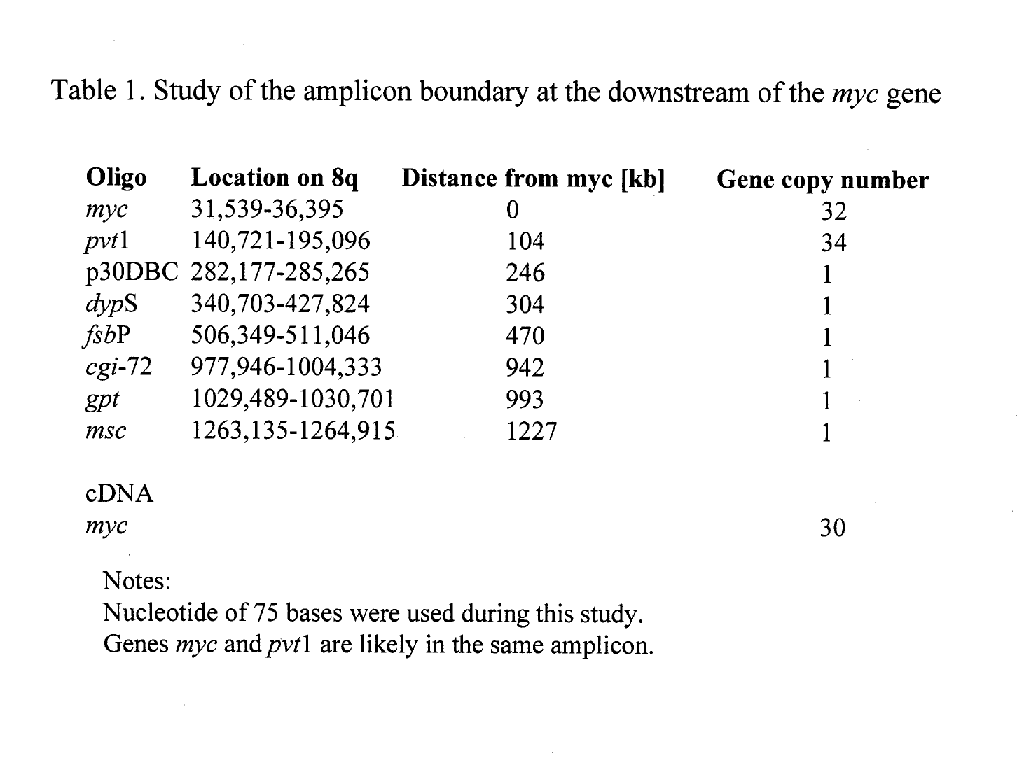

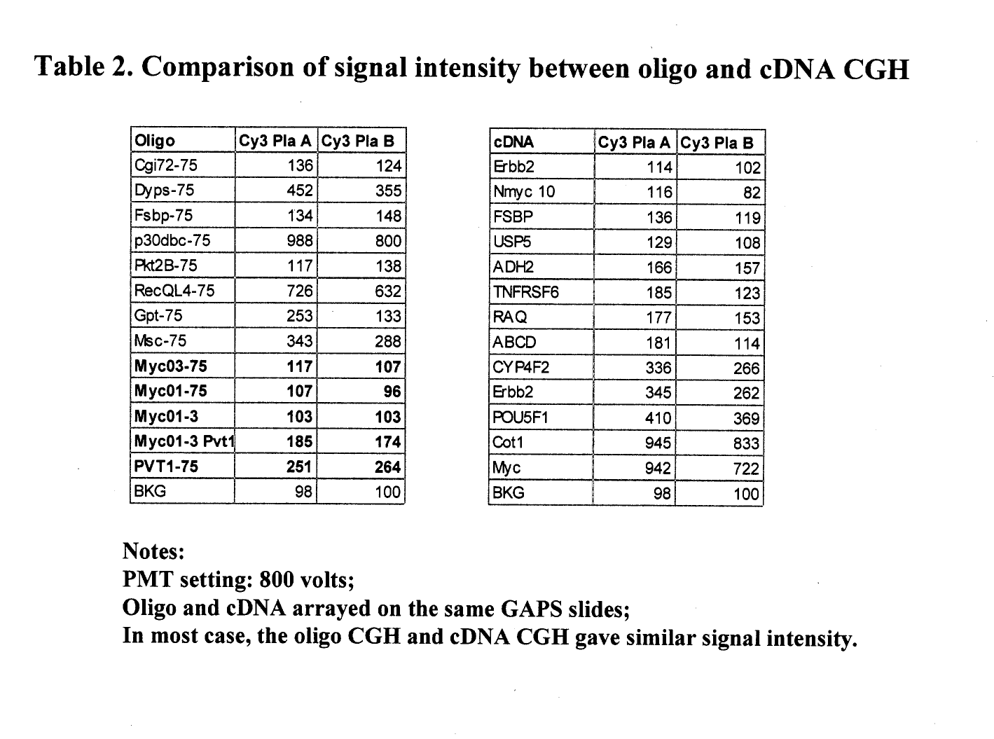

The genes of MYC (or cMYC) and PVT1 are located on the chromosome 8 (8q24). FIG. 2A is an image of an oligonucleotide array. FIG. 2B is a graph that represents gene copy numbers for the array of FIG. 1A. Table 1 summarizes data for oligonucleotide CGH. The data indicated the indicated that the MYC and PVT1 genes were both amplified about 30 fold in Colo320 cells. According to the human genome sequence, they are approximately 104 kilobases (kb) apart, and most likely located on the same amplicon, which spans greater than a 1 Mb sequence. Table 1 shows that using the inventive method, detection of gene copy numbers for the MYC-gene using an oligonucleotide array is comparable to the data obtained from CDNA arrays. Data from Table 1, also suggested that other genes (e.g. DYPS, FSPP, CGI-72, GPT, MSC and P30DBC on chromosome 8q are 246 kb to 1227 kb from the cMYC gene) did not show any copy number changes. Thus, detection of sequence copy changes by the present oligonucleotide-array CGH technique is highly specific. The oligonucleotide array CGH was also highly reproducible as the four subgrids show in FIG. 3. With some model cell lines, it was demonstrated that oligonucleotide arrays could provide meaningful data, presented in Table 2. Profiling gene expression from cancer cells indicated that the cMYC gene is also highly expressed in Colo320 cells, but not in BT 474 cells. This suggests that there is an agreement among gene dosage and gene expression level for the cMYC gene in Colo320 cells, as shown in FIGS. 4A and 4B. The data demonstrates that the combination of the present inventive oligonucleotide array-based CGH and high sensitive expression assay can precisely detect oncogenes (e.g., MYC gene).

To label genomic DNA, a 28-29 μL solution containing 4 μg of human gDNA and 3.6-4 μg of random hexamers was incubated at 95° C. for 5 minutes, briefly chilled on ice, and then added to a 11-12 μL solution containing approximately: 4 μL of 10×EcoPol buffer; 2 μL of 0.1 M DTT; and 1.5-3 μL of a dNTP mixture. The dNTP mixture consisted of 10 mM each of dGTP, dATP, dTTP, and 1 mM of dCTP; 2 μL of Cy3- or Cy5-dCTP at 1 mM (PerkinElmer, Boston, Mass.); and 1-1.2 μL of klenow fragment (New England Biolabs, Inc., Beverly, Mass.). The combined total 40 μL solution was incubated at 37° C. for about two hours. Probes were purified using a QIAquick PCR purification kit according to the manufacturer's instructions (Qiagen, Inc., Valencia, Calif.). The cDNA concentration and the amount of Cy3/Cy5 incorporation was measured on an Agilent 8453E UV-Vis spectrometer.

Arrays were prehybridized in the following solutions: a) 2×SSC/0.05% SDS/0.25% NaBH₄ at 42° C. for 20 minutes to reduce autofluorescence; b) rinse with 1×SSC; c) 1×SSC/0.2%BSA at room temperature for 5 minutes; d) 1×SSC for 5 minutes; e) 0.2×SSC for 2 minutes for 2 times. After the final wash, the slides were spin dried in a centrifuge at 2000 rpm for 1 minutes. Each array was hybridized with a solution consisting of 40% formamide, 2×SSC, 0.2 μg/μL poly A, 0.4 μg/μL human Cot-1 DNA, 0.2% BSA, and a given amount of labeled DNA.

For hybridization, 50 μL of the solution was delivered onto the array and then spread over the entire surface using a 24 mm×50 mm cover slip. The arrays were incubated overnight at 42° C. Post hybridization wash was started. First, to remove the cover slip, by immersing arrays in 2×SSC/0.05% SDS at 42° C. for 1 minute. The slides were then washed by soaking in 2×SSC/0.05% SDS at 42° C. for 5 minutes, repeated once more; 1×SSC at room temperature for 5 minutes, repeated once more; 0.2×SSC at room temperature for 2 minute, repeated once more. The slides were spin dried in a centrifuge at 2000 rpm for 1 minute.

A GenePix 4000A Array Scanner was used to obtain the Cy3/Cy5 fluorescence images using a PMT setting of 750-950 volts. All images were analyzed using the GenePix Pro 3.0 analysis software (Axon Instruments, Inc., Foster City, Calif.).

II. A second example illustrates the method for performing RNA expression analysis. As in CGH, the RNA from a sample tissue is compared to normal human RNA in a dual color hybridization that includes the following major steps. First, extract total RNA from a sample tissue. Synthesize cDNA probes from the total RNA by reverse transcription. Label the sample cDNA probes a green fluorescent dye (e.g., Cy3-dCTP) and reference nucleic sample with a red fluorescent dye (e.g., Cy5-dCTP). Mix the labeled cDNA probes and reference sample with Cot-1 DNA to suppress repeat sequences. Hybridize a pool of complementary cDNA probes to a DNA chip and remove unbound probes through washing. Image the DNA chip to determine green and red fluorescence intensity for each target spot. Analyze statistically the color normalization and ratios to determine relative level of abundance of mRNA.

The microarrays employed the experiments were fabricated by a high-throughput contact printing technology developed at Coming Incorporated. The technology allows for the simultaneous deposition of 1024 elements in 6 seconds to printing four grids on each GAPS-coated glass slide, whereby each slide contains 3392 human genes and 19 Bacillus subtilus genes, to serve as negative controls.

The following reagents were used: human total RNA (Clonetech); Superscript II reverse transcriptase, DTT, RNAse H, RNase A, formamide, semi-random hexamers, human Cot 1 DNA (Life Technologies); Cy3-dCTP, Cy5-dCTP (NEN), RNAse A (USB), QIAquick PCR purification kit (Qiagen), bovine serum albumin (BSA), dextran sulfate, nuclease-free water, and poly A (Sigma).

First, to label total RNA, about 5 μg of total human RNA was used during primer-annealing. To two 1.5 ml micro-centrifuge tubes, one for Cy3 labeling and one for Cy5 labeling, the following components were added.

| | |
|---|---|
| Cy3 | Cy5 |

| | | | |
|---|---|---|---|
| Total human RNA (5.0 μg/μL) | 1 | 1 | μL |
| Semi-random hexamer (3 μg/μL) | 1 | 1 | μL |
| Anchored dT (0.5 μg/μL) | 1 | 1 | μL |
| Nuclease-free Water | n | n | μL |
| Total volume | 22.5 | 22.5 | μL |

The RNA sample was then incubated at about 70° C. for 5 minutes, followed by a quick chill on ice.

Subsequently, a reverse transcription labeling mixture consisting of the following was added to each tube.

| | |
|---|---|
| Cy3 | Cy5 |

| | | | |
|---|---|---|---|
| 1) 5X Superscript II buffer (BRL) | 8 | 8 | μL |
| 2) DTT (100 mM) | 4 | 4 | μL |
| 3) dNTP mixture | 2 | 2 | μL |
| 4) Cy3-dCTP (1 mM) | 1.5 | 0 | μL |
| 5) Cy5-dCTP (1 mM) | 0 | 1.5 | μL |
| 6) RT Enzyme (BRL) | 2 | 2 | μL |
| Total volume | 17.5 | 17.5 | μL |

The dNTP mixture consists essentially of a mix of 10 μL of 100 mM dGTP, 10 μL of 100 mM dATP, 10 μL of 100 mM of dTTP and 10 μL of 10 mM of dCTP, 60 μL of RNase/DNase free water, total volume 100 μL. The reverse transcription labeling mixture was added to the tube with annealed RNA, and mixed by vortex for about 10 seconds and spun for 10 seconds. Reverse transcriptase was then added and mixed well. The RNA was then incubated first at room temperature for 10 minutes, then at 42° C. for 2 hours. About 1 μL of RNase H and about 0.25 ul of RNase A, were added to degrade the RNA and incubated at 37° C. for 15 minutes. Subsequently, the probe material was purified using Qiagen's PCR purification kit. Five volumes of buffer PB (˜200 μL) was added to one volume of the labeling reaction (˜40 μL) and mixed. A QIAquick spin column was then placed in a 2 ml collection tube. To bind DNA, the sample was applied to the QIAquick column and centrifuge for 60 seconds (14000 rcf) at RT (25° C.). To wash, about 600 μL of Buffer PE was added to the QIAquick column and centrifuged for 60 seconds (14000 rcf) at room temperature (˜20-25° C.). The wash was repeated for another 3 times. The flow-through was discarded and the QIAquick column was placed back in the same tube. The column was then centrifuged for an additional 60 seconds (14000 rcf) at room temperature (˜20-25° C.). To elute the cDNA probes, about 30 μL of 0.5X Buffer EB (5 mM Tris-Cl, pH 8.5) was added to the center of the QIAquick membrane, and the column was let to stand for about 1 minute. Then, the column was centrifuged for about 60 seconds (14000 rcf) at room temperature (25° C.). An elute volume of about 28 μL and the cDNA concentration and fluorescent dye incorporation (net 260, 280, 550, 650 OD with 480 OD for background subtraction) were measured. Using a Speed Vac, the volume of probe was reduced from 28 μL to about 5-8 μL.

A number of hybridization reagents were prepared. They comprised the following solutions: 1) a 2% solution of BSA consisting of 1 g of BSA in about 50 ml of Mili-Q water; 2) array pre-hybridization solution consisting of 2×SSC/0.05% SDS/0.2% BSA made from a mixture of 10 ml of 20×SSC, 10 ml of 2% BSA, 80 ml of water, 0.25 ml of 20% SDS; 3) an autofluorescence reduction solution consisting of 2×SSC/0.05% SDS/0.25% NaBH₄ made from a mixture of 100 ml of wash solution (2×SSC/0.05%SDS) added to 0.25-0.4 g NaBH₄; and an array post-hybridization wash solution consisting of (a) 2×SSC/0.05% SDS, (b) 1×SSC, (c) 0.2×SSC.

During the pre-hybridization stage, a microarray slide substrate was treated with a reducing agent, NaBH4, and prewashed. A GenePix 4000A (Axon Instruments) fluorescence scanner was used to scan the microarray to obtain an auto-fluorescent signal. The Cy3/Cy5 fluorescence images were obtained using a PMT setting of 750-900 volts. About 100 ml of the post-hybridization solution (a) (2X SSC/0.05% SDS) was added to a Coplin jar and the solution was warmed up gradually to about 42° C. in a water bath (takes about 20-30 min). When the substrate slide is ready, solution (a) was transferred to ajar that contained about 0.25-0.4 g of NaBH₄. The slide was then soaked in the Coplin jar for about 10 or 20-60 minutes at 42° C. Then the slide was transferred to a Coplin jar filled with 1×SSC, at room temperature for 30 seconds, and repeated twice more. The slide was then transferred to another Coplin jar filled with about 100 ml of freshly made pre-hybridization solution (2×SSC/0.05% SDS/0.2% BSA) that was already warmed up to 42° C. in a water bath (takes about 20-30 min) and incubated at 42° C. for 15 minutes. The slide was transferred to a Coplin jar filled with 1×SSC at room temperature for 1 minute, and again to a Coplin jar filled with 0.2×SSC at room temperature for 1 minute. This step was repeated twice more. The slide was then spin-dried at 2000 rpm for 1 minute at 25° C. Microarrays were then scanned again to examine the auto-fluorescence.

Two possible approaches were considered in preparing the hybridization solution for human cDNA or oligonucleotide array platforms. In one approach, the following were combined in a 1.5 ml microcentrifuge tube: 18 μL of 100% Formamide; 6 μL of 20×SSC; 10 μL of 30% detran sulfate; 1.2 μL of 5% SDS; x μL of Cy3 probe (5 μg of total RNA input); y μL of Cy5 probe (5 μg of total RNA input); z μL of Nuclease free water; 1 μL of 10 μg/μL of poly A; 6 μL of 1 μg/μL Cot 1 DNA. The probes were then denatured at 95° C. for 3 minutes, spun at room temperature for 30 seconds, and incubated at 42° C. for 2 minutes. Afterwards, 6 μL of 2% BSA was added to make a final total volume of 60 μL. The solution was mixed well and incubated at 42° C. before applied to the slide. In a second approach about 10-15 μL of 20% dextran sulfate was added to the solution recited above and processed in identical fashion.

Next the targets on the microarray were hybridized. About 60 μL of the hybridization solution was applied onto the microarray slide, about 4-6 drops, along the middle line of the array. A 24 mm×60 mm cover-slip was then placed onto the array, mindful to avoid bubbles. The slide was then placed in a hybridization chamber comprised of a sealed pipette-tip box with 5×SSC buffer on the bottom. The microarray is placed immediately into an incubator at 42° C. for overnight of about 14-20 hours.

After incubation, the microarray was washed, without permitting the microarray to dry between individual washes. The microarray was immerse immediately in 2×SSC/0.05% SDS, contained in a 1^{st} Coplin jar (jar #1), at 42° C. for 1 minutes. In a series of washes, the slide was then transferred into a 2^{nd} Coplin jar (jar #2), with 2×SSC/0.05% SDS at 42° C. for 5 minutes, into a 3^{rd} Coplin jar (jar #3) with 2×SSC/0.05% SDS at 42° C. for 5 minutes, to a 4^{th} Coplin jar Car #4) with 1×SSC wash solution at room temperature for 5 minutes, to a 5^{th} Coplin jar (jar #5) with 1×SSC wash solution at room temperature for 5 minutes, to a 6^{th} Coplin jar (jar #6) with 0.2×SSC wash solution at room temperature for 2 minutes. The last wash using 0.2×SSC wash solution at room temperature for 2 minutes was repeated twice in jars #7 and #8. The slide is finally pin-dried at 2000 rpm at 25° C. for 1 minute. The slide was stored in the dark before imaging.

Two image settings are suggested. Low PMT setting, where the brightest spot was close to saturation (65000 RFU) for each channel (Cy3 and Cy5). High PMT setting, where the top ˜5% of spots were saturated (>65000 RFU) for each color.

Assessment of High Throughput and High Quality Printing Technology

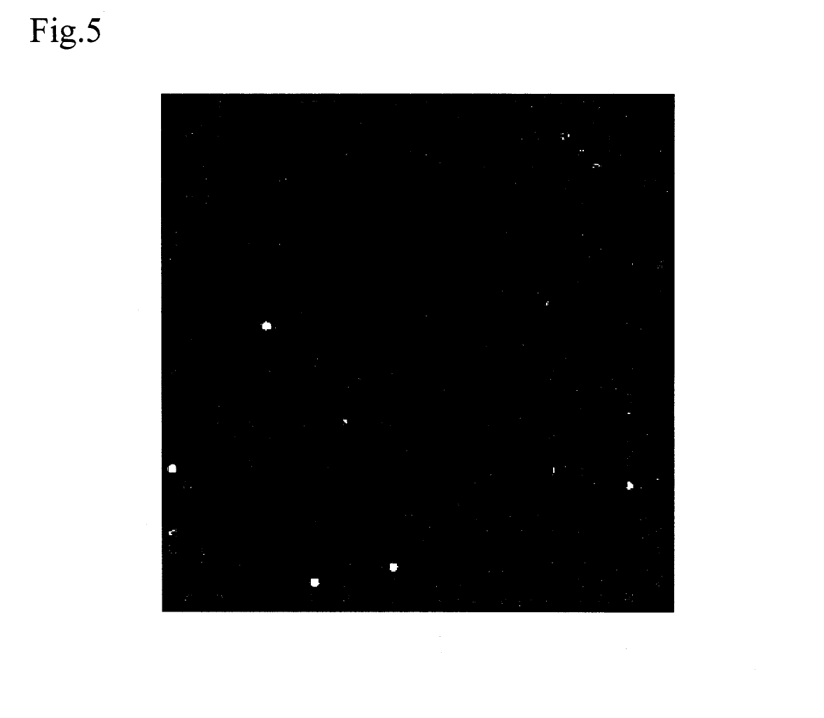

A novel, high-throughput contact printing technology developed at Coming Inc. showed uniform distribution of spots on glass slides as visualized by SYBR green staining as seen in FIG. 5. The printed slides were found to be of excellent quality and integrity and the spot morphology in 6K, 4K and 2K arrays was found to be homogeneous, confirming the high printing quality of Coring microarrays.

Probe Labeling with Random/Semi Random Primers

In general, CDNA microarrays typically contain PCR-amplified EST clones as targets, which contain poly-A regions of different lengths. Commonly, poly-dT primers are used for primer extension to label total eukaryotic RNA samples. Since the primer extension with poly dT or anchored poly-dT starts reverse transcription from the 3′ end of mRNA exclusively, the labeling near the 5′ end of RNA is not as efficient as near the 3′ end as a consequence of either the secondary structure of RNA, the length of the mRNA, or the fluorescent dyes and due to early termination of the transcription process. This could be problematic, especially for DNA oligonucleotide arrays. For a given gene the specific hybridization signal could be lower for targets located near its 5′ end than for targets near its 3′ end, which in turn could affect the ratios.

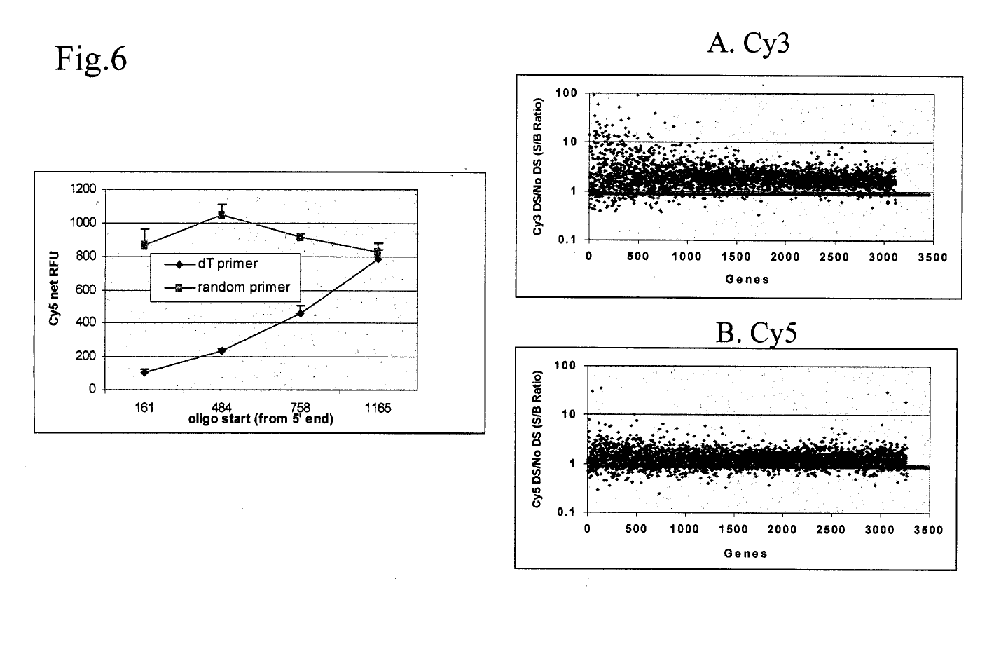

To demonstrate this point, a set of DNA targets for B. subtilis gene was deposited on an array. A sample of 1.2 kb B. subtilis RNA (with an engineered poly-A tail) was produced using in vitro transcription. For the set of tiling oligonucleotides, we have synthesized 4 oligonucleotides (60mers) to cover the whole length of the RNA molecule. Each oligonucleotide was 300-400 nt apart. These oligonucleotides were printed on GAPS slides as targets. RNA was labeled by reverse transcription with either poly-dT primer or semi-random primers. The hybridization results showed that both the Cy3 and Cy5 signal with poly-dT labeled probe is similar to random primer labeled probe near the 3′ end of RNA, indicating both primers work with similar efficiency. The hybridization signal, however, dropped significantly for the targets near the 5′-end with poly-dT primer labeled probe, as depicted in FIG. 6. This reflects the reduced transcription efficiency of the 5′ end compared to the 3′ end and reveals the advantage of using random primer over poly-dT primers during reverse transcription.

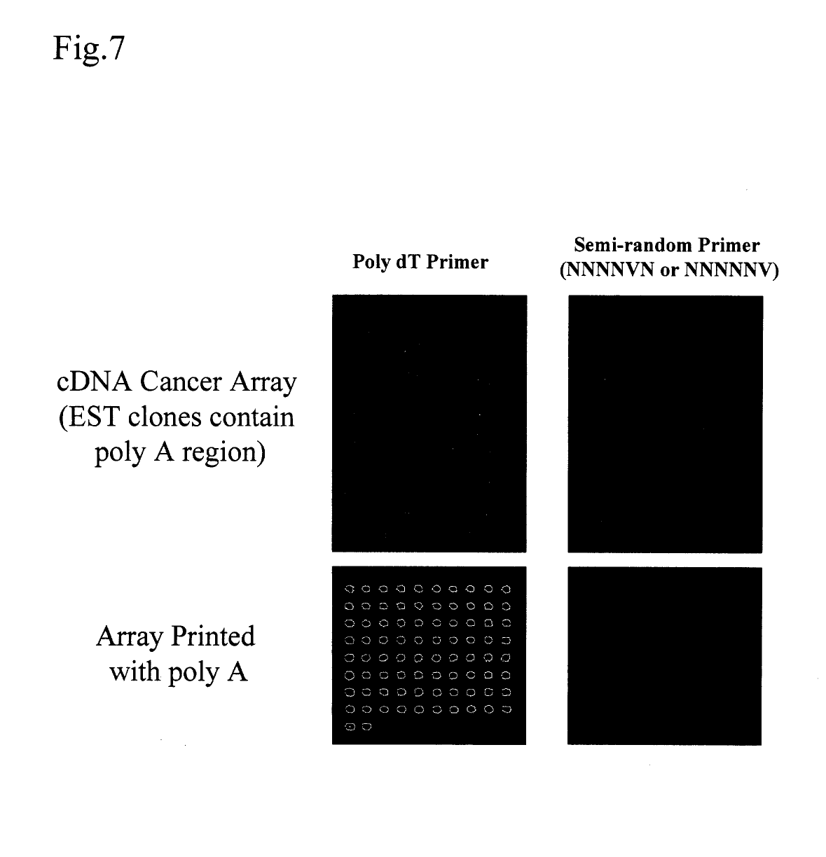

To improve the specificity of the probe, we also have used semi-random primers (NNNNVN, N=dA, or dG, or dC, or dT; V=dA, or dG, or dC) for total RNA or mRNA labeling. Compared to poly-dT primers, less primer extension occurs on poly-A region of the mRNA with semi-random primers. We printed poly A on GAPS slides and hybridized poly-A labeled probes with dTTP and Cy3/Cy5 dUTP using different primers (poly-dT, semi-random hexamers). As shown in FIG. 7, poly-dT primer labeled probe hybridized to CDNA and poly-A targets resulting in positive signals suggesting the potential for cross-hybridization. Interestingly, no hybridization signal was observed with semi-random primer labeled probe. Since the poly-A is labeled with less semi-random primers, it appears to improve the specificity of hybridization.

Hybridization Conditions

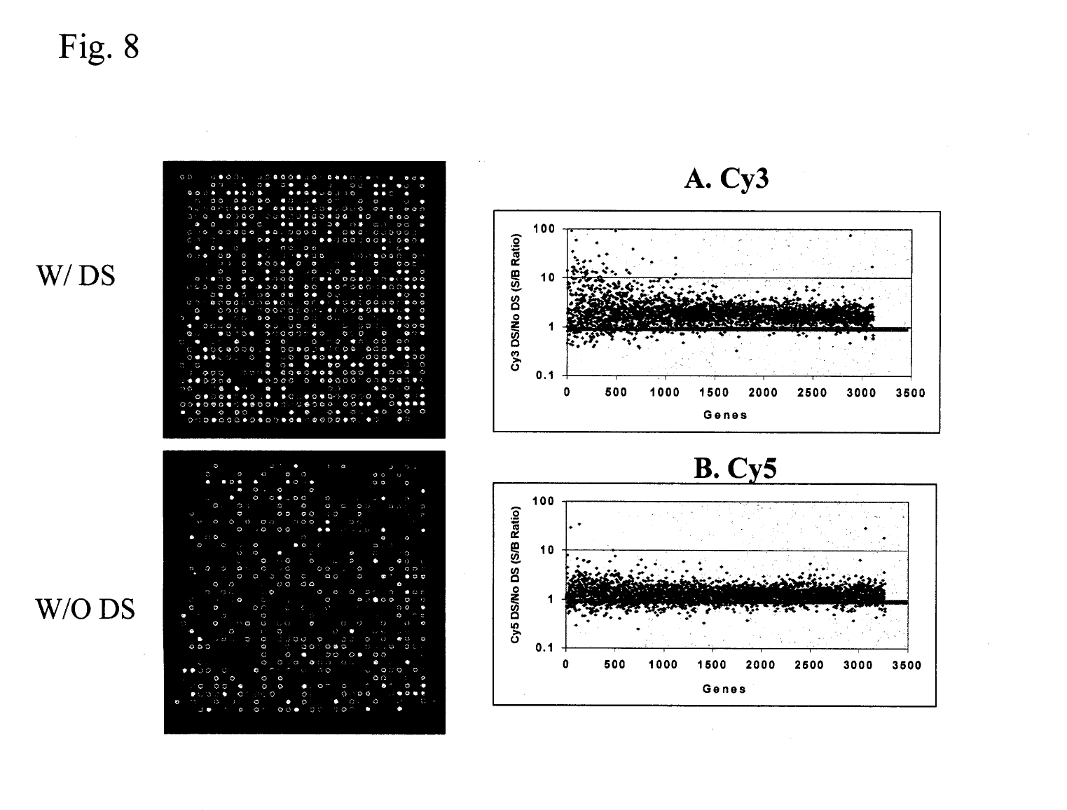

High specificity of discrimination for target molecules as well as low background signals are critical aspects in developing hybridization assays on microarrays. The performance of DNA array is severely compromised by auto-fluorescent signals that may originate from either the DNA spots or the slide surface to which the target DNA is immobilized. To eliminate false signals due to auto-fluorescence, we pre-treated DNA arrays with sodium borohydride (NaBH₄) solution, since we have previously demonstrated a dramatic reduction of auto-fluorescence on various slide surfaces. Dextran sulfate is commonly used in Northern blotting or Southern blotting to increase the hybridization signal. Few DNA-microarray assay methods, however, have suggested the use of dextran sulfate during hybridization. We found that the addition of dextran sulfate results in probe aggregation and precipitation at low temperature and high DNA concentrations, leading to discrimination, uneven and high fluorescent background. Also the addition of dextran sulfate reduces the stringency of hybridization, and as a result, decreases the specificity of hybridization. On the other hand, we also found that hybridization with BSA can significantly reduce non-specific biding of probe on GAPS slide surface. After evaluating these facts, we adjusted the dextran sulfate concentration to ˜4.5%, formamide concentration to ˜35%, reduced the salt concentration to ˜2×SSC and adjusted the hybridization temperature to 42° C. As shown in FIGS. 4A and 4B, the hybridization signals with dextran sulfate were found surprisingly to be significantly higher than those without dextran sulfate (FIG. 8A). Both the signal/background ratios of Cy3 and Cy5 were 2-10 fold higher with the addition of dextran sulfate (FIG. 8B). Our studies with Ficoll and PVP also suggested that there wasn't any significant effect on the reduction of background. The effect of blocking agents such as tRNA on background reduction was found to be insignificant. Genomic DNA did reduce background, but also reduced the hybridization signal, presumably by affecting the hybridization kinetics via non-specific binding with probes (data not shown).

High Sensitivity of Gene Expression Profiling

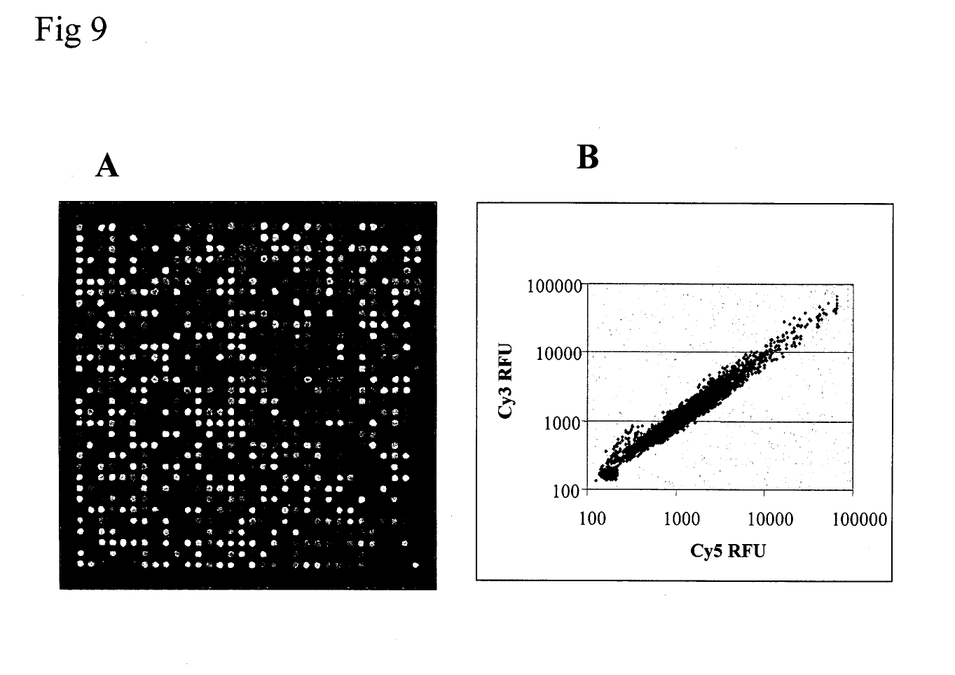

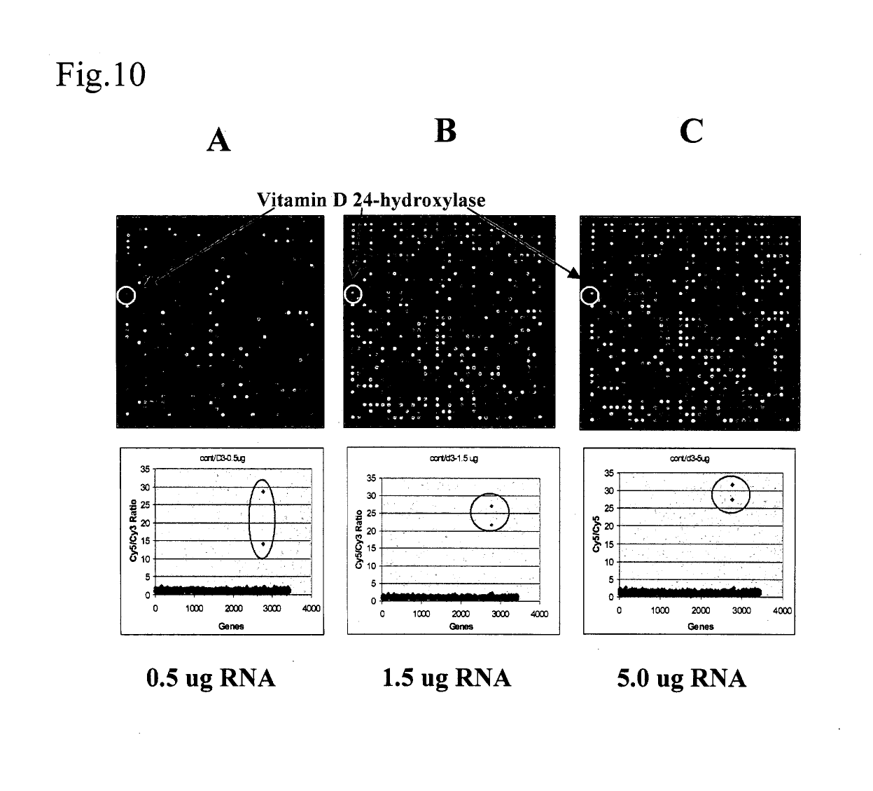

FIG. 9A shows one sub-grid of the Cy3 and Cy5 composite image of self-self hybridized yeast RNA onto a 6K yeast array. The plot of Cy5 vs. Cy3 signal shows broad dynamic range (>3 orders) and good correlation between two signals (R=0.99)(FIG. 9B). The small CV of ratio (9%) suggesting that it is able to detect 50% ratio changes (FIG. 9C). The sensitivity of assay was examined by hybridizing different amounts RNA to the arrays. Varying amounts of total RNA from breast cancer cells MCF-7 (0.5-5 ug) were labeled with Cy3 (untreated cells) and Cy5 (cells treated with vitamin D3 for 6 hours). Hybridization results showed that the gene expression profile remained quite consistent with RNA from 0.5 μg to 5 μg under improved assay conditions. As represented in FIG. 10, the up-regulation of vitamin D24 hydroxylase was observed repeatedly even with 0.5 μg of total RNA.

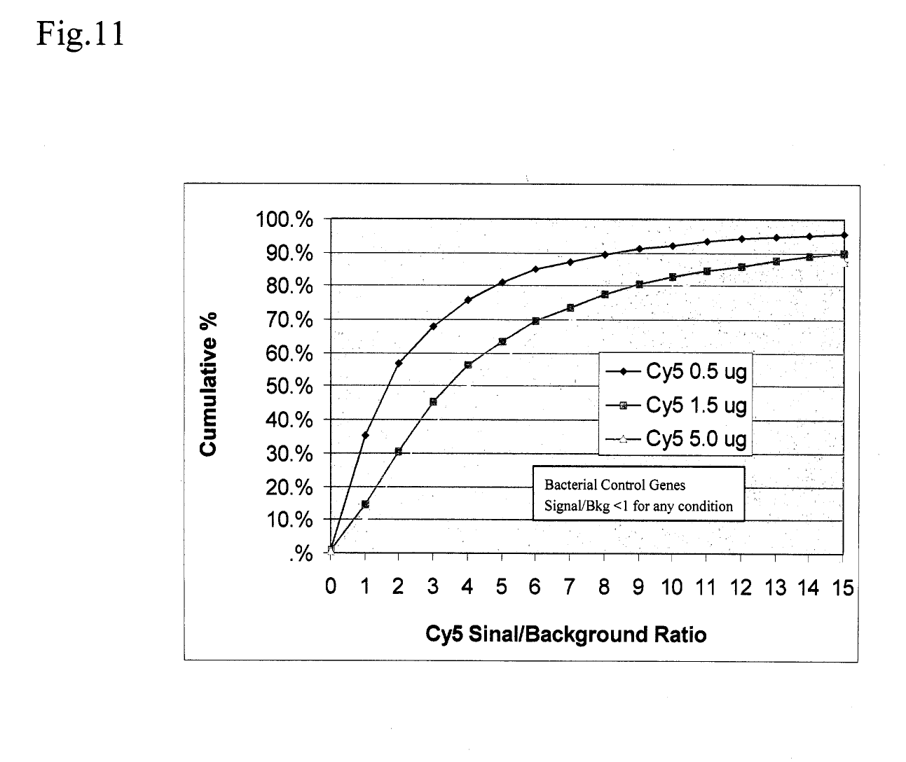

To calculate the number of positive elements for the varying amounts of RNA, we analyzed the signal to background ratio for all spots. FIG. 11 shows the plot of net Cy5 signal (spot RFU-local background RFU)/background ratio vs cumulative percentage of all genes. For the slide hybridized with 5 μg of total MCF RNA, 90% of targets have signal/background ratios greater than 1, and 75% of targets have signal/background ratio greater than 2. For the slide hybridized with 1.5 μg of total MCF RNA, 85% of targets have signal/background ratio greater than 1, 70% of target have signal/background ratio greater than 2. For the slide hybridized with 0.5 μg of total MCF RNA, 65% of targets have signal/background ratio greater than 1, 43% of targets have signal/background ratio greater than 2.

Validation of Positive Signals on Microarray by RT-PCR

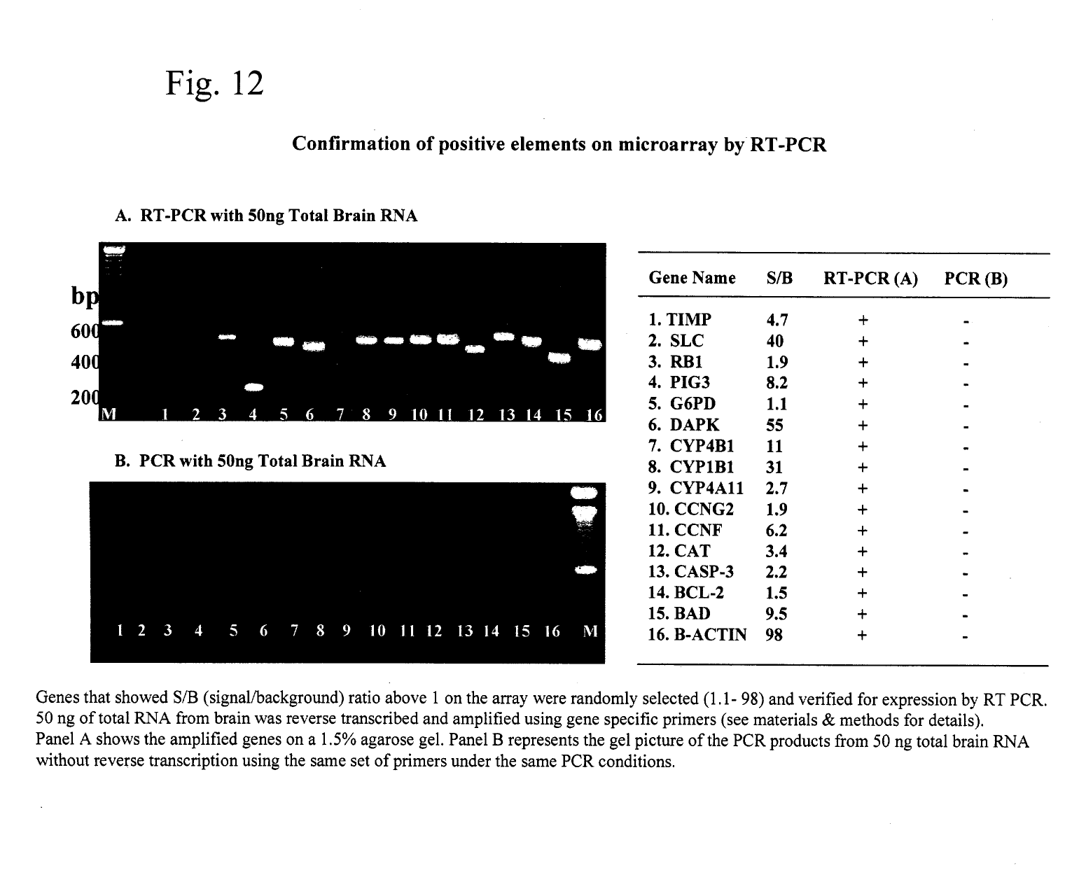

Gene specific RT-PCR experiments conducted on few selected genes which had Signal/Background ratios ranging from about 1.1 to about 98 confirmed the positive expression of genes even at Signal/Background ratio 1.1 on the microarray. FIG. 12 demonstrates this high specificity of our assay system clearly. A sensitive and specific hybridization method for expression profiling using CDNA microarrays is provided. Generally, the method for expression analysis consists of a series of steps starting from extraction of RNA followed by synthesis of labeled double stranded cDNA, a highly stringent hybridization assay and the detection of the labeled probe to the target DNA immobilized on the surface. Most of the methods that are currently being followed require mRNA for the synthesis of cDNA along with amplification of RNA by in vitro transcription. The major disadvantage in this is the significant loss of sample during mRNA extraction, resulting in the use of large amounts of cells or tissue as starting material. This eventually led to the incorporation of RNA amplification step in microarray analysis method, which is currently being used on a routine basis in many laboratories.

The protocol that we have optimized and reported here, is capable of detecting genes with copy numbers less than 1 per cell in 1×10⁵ cells using less than 5 μg of total RNA without tedious RNA amplification. This will greatly facilitate the use of cDNA arrays for expression analysis in small samples especially in molecular diagnostics. This is a significant improvement over the currently used methods which are able to detect 0.2 copies per cell using oligonucleotide-based arrays, and 3 copies per cell in 10⁶ cells using cDNA arrays. According to the method, labeling with random primers appears to generate high quality probes, which generated highly specific signals upon hybridization to target DNA on the surface. Non-specific signals due to non-specific binding of probes and auto-fluorescence were eliminated to a significant level (>90%) by the present stringent pre-hybridization and post-hybridization washing buffers. Self-Self hybridization experiments demonstrate the sensitivity of the assay system with dynamic range of signals greater than 3 logs, excellent co-relation between Cy3 and Cy5 (R=0.99) and CV less than 9% on inter and intra slide comparisons. RT-PCR experiments clearly demonstrated the high sensitivity of our assay system by confirming positive gene expression even at signal/background ratio at 1.1. Differential gene expression analysis with different concentrations of RNA starting from 0.5 μg to 5 μg RNA in breast cancer cells treated with vitamin D3 showed more or less similar gene expression patterns with respect to genes that are known to be up or down regulated. This enhanced specificity and sensitivity of the array system was successfully applied in comparative genomic hybridization studies to detect amplified genes in pathological samples. Hence, the CDNA arrays used according to the present invention and the hybridization conditions we have developed can out perform currently available methods, in terms of sensitivity and specificity with tremendous potential for single-cell, gene-expression analysis in the future.

Table 3 provides a summary of the method for performing RNA expression.

TABLE 3
Process

| | |
|---|---|
| Substrate Coating | Gama-aminopropylsilane (GAPS) |
| Ink | 80% ethylene glycol (EG) |
| Post array | NaBH4 treatment |
| | BSA blocking agent |
| RNA Labeling | 1-5 μg of total RNA with random, semi- |
| For each hybridization | random primers, and/or anchored poly-dT |
| Genomic DNA Labeling | 2-4 μg |
| Genomic DNA Hybridization | 4 μg of probe |
| | 2X SSC |
| | 0.05% SDS |
| | 40% Formamide |
| | 20 μg Cot 1 |
| | 10 μg poly-A |
| | 0.2% BSA |
| | in 50 μl, at 42° C. |
| RNA hybridization | 0.1-0.2 μg mRNA or 1-5 μg of total RNA |
| | probe |
| | 2X SSC |
| | 30% Formamide |
| | 6 μg Cot 1 |
| | 10 μg poly-A |
| | 0.2% BSA |
| | 0-6% dextran sulfate |
| | in 60 μl, at 42° C. |
| Final Wash | 0.2X SSC |

III. In an alternative manifestation of the method, DNA was extracted. DNA polymerase was applied for genomic DNA labeling during primer extension. In an experiment, a 28 μL solution containing 4 μg of human genomic DNA and 4 μg of random hexamers was incubated at 95° C. for 5 minutes, briefly chilled on ice and then added to a 12 μL solution containing 4 μL of 10×EcoPol buffer; 2 μL of 0.1 M DTT; 3 μL of a dNTP mixture consisting of 10 mM each dGTP, dATP, dTTP, and 1 mM of dCTP; 2 μL of Cy3- or Cy5-dCTP (1 mM, PerkinElmer, Boston, Mass.); and 1 μL of klenow fragment (New England Biolabs, Inc., Beverly, Mass.). The combined 40 μL solution was incubated at 37° C. for two hours. Probes were purified using a QIAquick PCR purification kit according to the manufacturer's instructions (Qiagen, Inc., Valencia, Calif.).

Arrays were prehybridized in the following solutions: 2×SSC/0.05% SDS/0.2% BSA at 42° C. for 20 minutes; 1×SSC at room temperature for 5 minutes, repeat once more; 0.2×SSC for 5 minutes, repeat once more. After the final wash, slides were spin dried in a centrifuge at 2000 rpm for 1 minute. A solution of: 20 μL 100% formamide; 5 μL of 20×SSC; 1 μL of 10 μg/μL poly A; 2 μL of 10 μg/μL human Cot-1 DNA; 1 μL of 5% SDS; 2-5 μL of Cy3 test DNA (˜4 μg); 2-5 μL of Cy5 reference DNA (˜4 μg) are combined in a 1.5 mL microcentrifuge tube and deionized (DI) water is added to bring the solution to a final volume of 50 μL.

To hybridize the probe sequences to the target sequences, one vortexed the hybridization solution, then microcentrifuged at 12,000 rpm for 5 seconds, incubated in an 95° C. water bath for 5 minutes in order to denature all DNAs, microcentrifuged at 12,000 rpm for 1 minute at room temperature, 5 μl of 2% BSA solution was added to the hybridization solution and mixed well. The entire hybridization mixture (50 μL final volume) was applied to the array and spread over the entire surface using a 24 mm×50 mm cover slip, and the DNA chip(s) was incubated at 42° C. in the dark overnight (15-18 hours).

After hybridization, the microarray was washed in a series of 8 Coplin jars. Initially, one wash was performed in 2×SSC/0.05% SDS at 42° C. for 1 minute to remove the cover slip; 2 washes in 2×SSC/0.05% SDS at 42° C. for 5 minutes; 2 washes in 1×SSC at for 5 minutes each at room temperature; 3 washes in 0.2×SSC for 2 minutes each at room temperature.

Remove biochips from last wash, and dry biochips in a centrifuge at 2000 rpm for 1 min. Adequate washing is vital for good assay performance. When transferring biochips from one bath to the next, it is helpful to agitate the biochip with an up and down and back and forth motion to ensure proper washing. Do not allow chips to dry between washes. Any fingerprints, dust or lint on the chip can affect the image, therefore, remove any dust with a clean source of compressed air.

A GenePix 4000A (or 4000B) Array Scanner was used to obtain the Cy3/Cy5 fluorescence images using a PMT setting of 750-950 volts. All images were analyzed using the GenePix Pro 3.0 analysis software (Axon Instruments, Inc., Foster City, Calif.).

The cancer array, such as that manufactured by Corning, usually contains approximately 2000 genes in 2 replicates and is designed for measuring gene expression profiles and genomic amplifications in tumor DNA. The hybridizations were carried out with a mixture of total human reference DNA (labeled with Cy3) and DNA extracted from the tumor cell line Colo320 (labeled with Cy5). This cell line was chosen because of the amplification characteristics of the cMYC oncogene, which can be readily seen in FIG. 4. The normalized Test/Reference fluorescent signal ratios for the cMYC target reveals a 32 fold amplification, which agrees well with the literature (28-35 fold). In addition the data suggest so far unreported amplifications for genes abcD1 (7×), mcl1 (12×), and pou5F1 (6×) (FIG. 5), located on chromosomes Xq28, 1q21 and 6q21, respectively.

Gene-copy number changes for several genes have been well characterized in the breast cancer cell line BT474. Analysis of DNA extracted from this cell line with our cDNA arrays shows the known amplification of ERBB2 (6×). As seen in FIG. 6, the finding of a 6 fold amplification for the gene CYP24 confirms a recent report that identifies the CYP24 gene as an oncogene candidate, located on an approximately 2-Mb amplified (8-12×) region of chromosome 20q13.2.

Although the present invention has been described generally and by way of examples, those skilled in the art will understand that the invention is not limited to the embodiments specifically disclosed, and that various modifications and variations can be made without departing from the spirit and scope of the invention. The inventive methods described above can be employed in oligonucleotide-array-based CGH studies for both eukaryotic and prokaryotic organisms to provide accurate diagnosis of gene dosage variation for genetic diseases and in study of the relationship of gene dosage and disease phenotype, on the scale of an entire genome. The methods also can be easily adapted to gene expression profiling as described above. Therefore, unless changes otherwise depart from the scope of the invention as defined by the following claims, they should be construed as included herein.

We claim:

1. A method for detecting genetic aberrations, the method comprising: a) using a protocol selected for either performing comparative genomic hybridization (CGH) on a nucleic acid array, or performing gene-expression analysis using RNA; b) performing CGH, or optionally performing gene-expression, respectively.

2. The method according to claim 1, wherein the protocol for performing CGH comprises the steps of:
a) reducing autofluorescence on a substrate containing an array of oligonucleotides; the autofluorescence reducing step further comprising:
(1) providing a substrate having a first surface with a functional group for binding unmodified oligonucleotides;
(2) arraying a set of target oligonucleotides onto said first surface;
(3) treating at least a portion of the first surface with a reducing agent;
b) applying an amount of genomic DNA (gDNA) probes, without amplifying said gDNA, of about 10 μg or less;
c) labeling gDNA from a test sample and a reference sample with a first fluorescent dye and a second fluorescent dye, respectively;
d) pretreating said first surface of said substrate with a blocking reagent to reduce non-specific binding of said gDNA probes to said first surface or target oligonucleotides;
e) increasing local concentration of gDNA probes to promote hybridization efficiency and optimizing stringency to promote specificity by means of a predetermined hybridization mixture;
f) hybridizing said gDNA probes to said target oligonucleotides;
g) treating again, during hybridization, said first surface of said substrate with said blocking reagent to further minimize non-specific binding of gDNA probes to said target oligonucleotides or the first surface;
h) imaging the relative fluorescence intensity of said first and second fluorescent dyes.

3. The method according to claim 1, wherein the protocol for performing gene-expression comprises the steps of:
a) reducing autofluorescence on a substrate containing an array of biomolecules;
the autofluorescence reducing step further comprising:
(1) providing a substrate having a first surface with a functional group for binding of an unmodified biomolecule;
(2) arraying a set of target biomolecules onto said first surface;
(3) treating at least a portion of the first surface with a reducing agent;
b) applying an amount of either total RNA or mRNA, without amplification, of about 10 μg or less.
c) labeling by fluorescent means a number of cDNA probes, which are generated by reverse transcription from either said total RNA or mRNA using either random primers, semi-random primers, anchored dT, or a combination thereof;
d) pretreating said first surface of said substrate with a blocking reagent to reduce non-specific binding of said cDNA probes to said first surface or target biomolecules;
e) increasing local concentration of cDNA probes to promote hybridization efficiency and optimizing stringency to promote specificity by means of a predetermined hybridization mixture;
f) hybridizing a pool of complementary cDNA probes to the target biomolecules;
g) treating again during hybridization said first surface of said substrate with said blocking reagent to reduce non-specific binding of said cDNA probes to said first surface or target biomolecules;
h) imaging said first surface to determine the relative fluorescence ratio of hybridized cDNA probes and target biomolecules.
i) analyzing said fluorescence ratio to determine relative gene copy numbers.

4. The method according to claim 2, wherein said imaging step determines a ratio of said first and second fluorescent dyes to represent a copy number of a gene.

5. A method for performing comparative genomic hybridization (CGH) on a oligonucleotide-based DNA array, the method comprising the steps of:
a) providing a substrate containing an array of oligonucleotides;
b) reducing autofluorescence of the oligonucleotides;
c) applying an amount of genomic DNA (gDNA) probes, without amplifying said gDNA, of about 10 μg or less;
d) labeling gDNA from a test sample and a reference sample with a first fluorescent dye and a second fluorescent dye, respectively;
e) pretreating a first surface of said substrate with a blocking reagent to reduce non-specific binding of said gDNA probes to said first surface or target oligonucleotides;
f) hybridizing said gDNA probes to said target oligonucleotides;
g) imaging the relative fluorescence intensity of said first and second fluorescent dyes to determine a ratio of said first and second fluorescent dyes to represent a copy number of a gene.

6. The method according to claim 5, wherein the method further comprises eliminating autofluorescence of said oligonucleotides according to the steps of: providing a substrate having a first surface with a functional group for binding unmodified oligonucleotides; arraying a set of target oligonucleotides onto said first surface; treating at least a portion of the first surface with a reducing agent.

7. The method according to claim 5, wherein the method further comprises increasing local concentration of gDNA probes to promote hybridization efficiency and optimizing stringency to promote specificity by means of a predetermined hybridization mixture.

8. The method according to claim 5, wherein the method further comprises treating again, during hybridization, said first surface of said substrate with said blocking reagent.

9. The method according to claim 5, wherein the amount of gDNA applied, without amplifying said gDNA, is about 1-10 μg.

10. The method according to claim 5, wherein the amount of gDNA applied, without amplifying said gDNA, is about 5 μg or less.

11. The method according to claim 5, wherein said oligonucleotides have a length of about 15 bases to about 110 bases.

12. The method according to claim 5, wherein said oligonucleotides have a length between about 40 bases to about 100 bases.

13. The method according to claim 5, wherein said gDNA is not amplified by PCR amplification.

14. An oligonucleotide-based microarray for performing genetic-aberration analysis using total genomic DNA without amplifying, according to the method of claim 5.

15. A method for performing RNA expression analysis, the method comprising the steps of:
a) reducing autofluorescence on a substrate containing an array of biomolecules;
b) applying an amount of either total RNA or mRNA, without amplification, of about 10 μg or less.
c) labeling by fluorescent means a number of cDNA probes, which are generated by reverse transcription from either said total RNA or mRNA using either random primers, semi-random primers, anchored dT, or a combination thereof;
d) pretreating a first surface of said substrate with a blocking reagent to reduce non-specific binding of said cDNA probes to said target biomolecules or first surface;
e) hybridizing a pool of complementary cDNA probes to the target biomolecules;
f) analyzing said fluorescence ratio to determine relative differential gene expression levels.

16. The method according to claim 15, wherein the method further comprises the autofluorescence reducing step further comprising: providing a substrate having a first surface with a functional group for binding of an unmodified biomolecule; arraying a set of target biomolecules onto said first surface; treating at least a portion of the first surface with a reducing agent.

17. The method according to claim 15, wherein the method further comprises increasing local concentration of cDNA probes to promote hybridization efficiency and optimizing stringency to promote specificity by means of a predetermined hybridization mixture.

18. The method according to claim 15, wherein the method further comprises treating again, during hybridization, said first surface of said substrate with said blocking reagent.

19. The method according to claim 15, wherein the method further comprises imaging said first surface to determine the relative fluorescence ratio of hybridized cDNA probes and target biomolecules.

20. The method according to claim 15, wherein said amount of either total RNA or mRNA applied, without amplification, is about 5 μg or less.

21. The method according to claim 15, wherein said amount of mRNA applied, without amplification, is about 0.2 μg or less.

22. The method according to claim 15, wherein said cDNA probes have a length of between about 100 bases to about 7 kilobases.

23. The method according to claim 22, wherein said CDNA probes have a length of between about 400 bases to about 5 kilobases.

24. A microarray for performing gene expression using total RNA, mRNA, aRNA, cRNA, or other RNA, without need for amplification, according to the method of claim 15.
